(19) United States
(12) Patent Application Publication
(10) Pub. No.: US 2021/0370022 A1
(43) Pub. Date: Dec. 2, 2021

(54) ANCHORING STRAIN RELIEF MEMBER

(71) Applicant: Incept, LLC, Lexington, MA (US)

(72) Inventors: Ryan McNally Bean, Groton, MA (US); Raymond Lareau, Westford, MA (US)

(21) Appl. No.: 16/886,099

(22) Filed: May 28, 2020

(51) Int. Cl. A61M 25/00; A61M 25/09

(52) U.S. Cl. CPC A61M 25/0075; A61M 25/09; A61M 2025/091; A61M 2205/0216; A61M 2025/0079; A61M 25/0097

(57) ABSTRACT

An anchor strain relief member provides resistance to disengagement from a hemostatic valve due to forces tending to force the catheter in a proximal direction. The anchoring strain relief member is distal to a hub, joined to the catheter outer surface, and comprises a sealing portion that has at least one ridge that has a ridge tip and a ridge height defined by a distance from the ridge tip to the catheter central axis. Methods of forming a nested catheter system are described using a catheter with an anchor strain relief as the inner catheter for the nested catheter set. Systems of a hemostatic valve and a suitable catheter with an anchor strain relief member can provide for desired assemblies of components.

### TECHNICAL FIELD

The Technical Field relates to a strain relief member for a medical catheter, in particular a strain relief member that has a surface for sealing and anchoring against a compressible material such as an elastomeric member. Catheters, methods, and systems for use with the strain relief member are also in the technical field.

### BACKGROUND

Medical catheters conventionally have a hub attached to a catheter shaft and a strain relief member joined to the shaft immediately distal to the hub, typically adjacent to, or overlapping with, the hub or in continuity with the hub. The hub is a connector that is connectable to fittings of a delivery system. The catheter provides passage of materials between the delivery system, the hub, and a lumen of the catheter. The catheter terminates at a distal tip. The delivery system may further provide for infusion, or alternatively removal and/or withdrawal of materials via the catheter lumen.

The strain relief member is designed to prevent collapse of a catheter shaft under lateral (bending) forces. And it is designed to prevent undue bending of the catheter shaft at or near the hub/tube junction. The hub is typically rigid relative to the catheter shaft and lateral forces tend to concentrate to create kinks in the shaft. The strain relief member distributes lateral forces so that they do not kink or otherwise unduly bend the catheter shaft. Besides designing for lateral forces, a strain relief member should be designed to avoid breakage of the member or its separation from the catheter shaft and/or hub.

### SUMMARY OF THE INVENTION

In a first aspect, the invention pertains to a medical catheter that comprises a strain relief member that provide a gripping surface in a sealing area to provide a resistance to movement and radial compression while promoting a seal when compressed against a deformable material. Strain relief members are not conventionally used or designed to provide a seal and a gripping surface in a sealing area. Certain embodiments include a strain relief member that has a sealing area that includes a plurality of ridges. This design has numerous advantages that become evident after reading the disclosure provided herein.

An embodiment of the invention is a medical catheter having a proximal end and a distal end, the catheter comprising a catheter shaft having catheter lumen(s), a catheter central axis, a catheter inner surface, and a catheter outer surface separated from the catheter inner surface by a catheter wall thickness, a hub attached to the proximal end of the catheter shaft, and an anchoring strain relief member distal to the hub, sealingly joined to the catheter outer surface, and comprising a monolithic sealing portion that comprises a plurality of ridges that each have a ridge tip and a ridge height defined by a distance from the ridge tip to the catheter central axis, the distance being measured perpendicular to the central axis. Embodiments include, e.g., a monolithic sealing portion having no taper or an appropriate taper. Uses include a use of the medical catheter for delivery of a substance, e.g., to treat or diagnose a disease or administer a therapy. In such uses, the monolithic sealing portion provides a seal against an elastomeric circumferentially sealing member (e.g. a hemostatic valve, such as a Tuohy-Borst Adapter).

An embodiment of the invention is a method of assembling a coaxial catheter system comprising the step of providing an outer catheter that comprises an outer catheter hub and an outer catheter shaft comprising an outer catheter lumen, an outer catheter inner surface, and an outer catheter outer surface, with the an outer catheter hub being connected to an outer catheter shaft to provide fluid communication between the outer catheter hub and the outer catheter shaft; providing an inner catheter that comprises an inner catheter hub, an anchoring strain relief member, and an inner catheter shaft comprising an inner catheter lumen with a central axis, an inner catheter inner surface, and an inner catheter outer surface, with the inner catheter hub being connected to the inner catheter shaft to provide fluid communication between the inner catheter hub and the inner catheter shaft, with the anchoring strain relief member being sealingly joined to the inner catheter outer surface; providing a connector that comprises a first opening and an elastomeric sealing member, with the sealing member providing a seal across the first opening; attaching the connector to the outer catheter hub in fluid communication with the outer catheter lumen and with a second opening between the connector and the outer catheter lumen, passing the inner catheter shaft through the first opening and the sealing member and into the outer catheter shaft lumen, with the connector being in fluid communication through the second opening with a annulus formed between the inner catheter outer surface and the outer catheter inner surface, and positioning a sealing portion of the strain relief member within the sealing member, with the sealing member engaged to press against the portion of the strain relief member to establish a seal.

An embodiment of the invention is a system or a kit comprising an elastomeric circumferentially sealing member of a Tuohy-Borst Adapter or other hemostatic valve,and a medical catheter comprising an anchoring strain relief member wherein the elastomeric circumferentially sealing member provides a seal around the catheter when the a portion of the anchoring strain relief member is positioned within an elastomeric sealing member of the Tuohy-Borst Adapter. The system or kit may have a proximal end and a distal end, the catheter comprising a catheter shaft having a catheter lumen, a catheter central axis, a catheter inner surface, and a catheter outer surface separated from the catheter inner surface by a catheter wall thickness, a hub attached to the proximal end of the catheter shaft, and the anchoring strain relief member is distal to the hub, sealingly joined to the catheter outer surface, and comprises a monolithic anchoring portion that comprises a plurality of ridges that each have a ridge tip that has a ridge height as defined by a distance from the ridge tip to the catheter central axis, the distance being perpendicular to the central axis.

In a further aspect, the invention pertains to a medical catheter having a proximal end and a distal end, the catheter comprising a catheter shaft having a catheter lumen, a catheter inner surface, and a catheter outer surface separated from the catheter inner surface by a catheter wall thickness, a hub attached to the proximal end of the catheter shaft, and an anchoring strain relief member distal to the hub, joined to the catheter outer surface. The anchoring strain relief member can comprise a sealing portion that comprises at least one ridge that has a ridge tip and a ridge height defined by a distance from the ridge tip to the catheter central axis, the distance being measured perpendicular to the central axis. Generally, the ridge forms a flow barrier between the catheter outer surface and the top of the ridge and if the sealing portion comprises a plurality of ridges each having a ridge tip and a ridge height, then a set of the ridge tips have no taper, a reverse taper, or no more than a 5 degree forward taper in a proximal to distal direction.

In another aspect, the invention pertains to a method of assembling a nested catheter system comprising:

providing an outer catheter that comprises:
an outer catheter hub and an outer catheter shaft comprising an outer catheter lumen, an outer catheter inner surface, and an outer catheter outer surface, with the outer catheter hub being connected to the an outer catheter shaft to provide fluid communication between the outer catheter hub and the outer catheter lumen;

providing an inner catheter that comprises:
an inner catheter hub, an anchoring strain relief member, and an inner catheter shaft comprising an inner catheter lumen with a central axis, an inner catheter inner surface, and an inner catheter outer surface, with the inner catheter hub being connected to the inner catheter shaft to provide fluid communication between the inner catheter hub and the inner catheter lumen, with the anchoring strain relief member being sealingly joined to the inner catheter outer surface;

providing a connector that comprises a first opening and an elastomeric sealing member, with the sealing member providing a seal across the first opening;

attaching the connector to the outer catheter hub in fluid communication with the outer catheter lumen and with a second opening between the connector and the outer catheter lumen,

passing the inner catheter shaft through the first opening and the sealing member and into the outer catheter shaft lumen, with the connector being in fluid communication through the second opening with a annulus formed between the inner catheter outer surface and the outer catheter inner surface, and

positioning a sealing portion of the strain relief member within the sealing member, with the sealing member pressing against the portion of the strain relief member to establish a seal.

In some aspects, the invention pertains to a system comprising a hemostatic valve and a medical catheter comprising an anchoring strain relief member comprising an elastomeric polymer and having a sealing portion. The hemostatic valve comprises a connector and a sealing member, and the sealing portion of the anchoring strain relief member can be engaged by the sealing member of the hemostatic valve to form a fluid tight seal.

### BRIEF DESCRIPTION OF THE DRAWINGS

FIG. 1A is an elevated side view depicting an embodiment of a catheter having an anchoring strain relief member;

FIG. 1B is an enlarged view of a longitudinal cross-section the anchoring strain relief member indicated by circle B in FIG. 1A;

FIG. 2 is an elevated side view depicting an alternative embodiment of a catheter having an anchoring strain relief member;

FIG. 3A is an elevated side view depicting an alternative embodiment of a catheter having an anchoring strain relief member;

FIG. 3B is a first embodiment of a ridge as viewed in a cross-sectional view taken along line B-B of FIG. 3A;

FIG. 3C is a second embodiment of a ridge as viewed in a cross-sectional view taken along line C-C of FIG. 3A;

FIG. 4 is an elevated side view depicting an alternative embodiment of a catheter having an anchoring strain relief member;

FIG. 5A is an elevated side view depicting an alternative embodiment of a catheter having an anchoring strain relief member;

FIG. 5B is a cross-sectional view taken along line B-B of FIG. 5A;

FIG. 6 is an elevated side view of an alternative embodiment of a catheter having an anchoring strain relief member with a reverse taper;

FIG. 7 is an elevated side view depicting an alternative embodiment of a catheter having an anchoring strain relief member with ridge tips defining a reverse taper;

FIG. 8 is an elevated side view depicting an alternative embodiment of a catheter having an anchoring strain relief member with a plurality of ridges defined by a plurality of notches;

FIG. 9A is a perspective view depicting an alternative embodiment of a catheter having an anchoring strain relief member;

FIG. 9B is an enlarged perspective view of the embodiment of FIG. 9A;

FIG. 10A is a side elevated view of the embodiment of FIG. 9A;

FIG. 10B is an elevated end view of the embodiment presented in FIG. 10A;

FIG. 11A is a top view of the embodiment of FIG. 9A;

FIG. 11B is an elevated end view of the embodiment presented in FIG. 11A;

FIG. 12A is an elevated side view depicting an alternative embodiment of a catheter having an anchoring strain relief member;

FIG. 12B is a cross-sectional view taken along section A-A of FIG. 12A;

FIG. 12C is a cross-sectional view taken along section B-B of FIG. 12A;

FIG. 12D is a cross-sectional view taken along section C-C of FIG. 12A;

FIG. 13A is an elevated side view of an alternative embodiment of a catheter having an anchoring strain relief member;

FIG. 13B is a cross-sectional view taken along section D-D of FIG. 13A;

FIG. 13C is a cross-sectional view taken along section E-E of FIG. 13A;

FIG. 14A is a plan view illustration of a delivery system incorporating coaxial catheters;

FIG. 14B is a plan view illustration of the embodiment of FIG. 14A after assembly;

FIG. 15A is a plot of experimental results showing force to dislodge an anchoring strain relief member during a backpres sure test;

FIG. 15B is a plot of the same backpressure test as used for FIG. 15A showing results for a conventional strain relief member;

FIG. 16A is a plot of experimental results showing a pull-out force required to move an anchoring strain relief member in a sealing position in a Tuohy-Borst Adapter; and

FIG. 16B is a plot of experimental results for a conventional strain relief sheath in the same pull-out force test as FIG. 16A.

### DETAILED DESCRIPTION

An embodiment of an anchoring strain relief member comprises a strain relief member having a surface suited to gripping and sealing. The member can have one or more generally a plurality of ridges that project from the member that can engage a deformable sealing member that is compressed against the anchoring strain relief member. The term ridge refers to a structure on the strain relief member that projects from the member relative to its immediate surroundings. A ridge can protect against proximal disengagement of the catheter from a hemostatic valve through providing an anchoring surface to engage an elastomeric sealing member and/or to provide a physical backstop or barrier to stop disengagement. The anchoring strain relief member has a sealing portion that provides a sealing surface when engaged with a sealing member, such as a sealing member of a hemostatic valve. The sealing portion providing the sealing surface may be made of a semi-rigid unit, for example, a single molded plastic piece or a single piece overmolded on a catheter, or the sealing portion can be assembled from a number of pieces. The sealing portion generally has effectively no taper, a negative taper in a proximal to distal direction, or a positive taper with no more than about 5 degree of taper.

The catheters with the anchoring strain relief members are particularly useful for the delivery of a second lumen through a larger catheter. The resulting nested catheter system provides two lumens, which may be, but are not necessarily, coaxial. The outer catheter can be attached to a fitting at a proximal hub, and the fitting comprises a suitable connector to attach to the catheter hub and a hemostatic valve providing the sealing member to engage the sealing portion of the anchoring strain relief member of the catheter. An embodiment of a delivery system is described below in which a dual channel delivery device delivers two chemical components through the separate lumen of a nested catheter system for combining at the distal end generally within a patient.

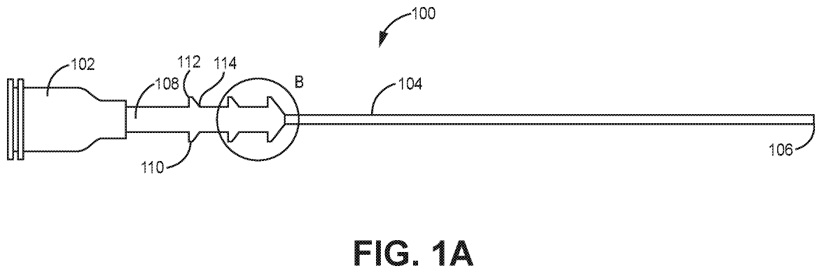

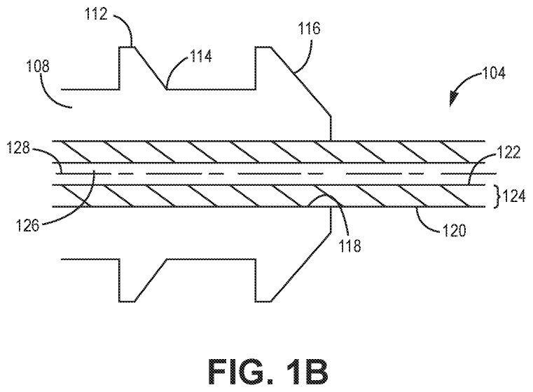

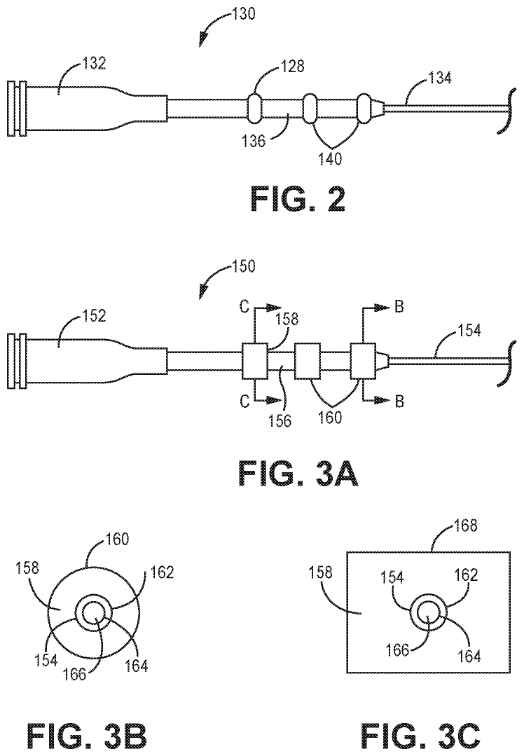

FIG. 1A depicts catheter 100 having hub 102, catheter shaft 104 having a distal tip 106, and an anchoring strain relief member 108 with barbs 110 having tips 112 proximal to barb bases 114. FIG. 1B is an enlarged view of area B of FIG. 1B, depicting strain relief member 108 top surface 116 and bottom surface 118. Catheter shaft 104 has outer surface 120 and inner surface 122 separated by wall 124, and lumen 126 with center axis 128. The gaps between bards 110 and proximal to the most proximal barb 110 can functions as notches, which can be engaged with an elastic sealing member of a hemostatic valve with the barbs then acting as backstops for any movement of the catheter within the valve. Similar implicit functions follow for the structures in the following FIGS. 2-5B.

FIG. 2 depicts catheter 130 having hub 132, catheter shaft 134 and an anchoring strain relief member 136 with rounded rings 138 having tips 140. FIG. 3A depicts catheter 150 having hub 152, catheter shaft 154 and an anchoring strain relief member 156 with flat rings 158 having tips 160. FIG. 3B is cross-sectional view of a first embodiment of flat rings 158 that have a cylindrical surface for tips 160, with catheter shaft 154 top surface 162 being directly joined to flat ring 158. Catheter shaft 154 has inner surface 164 that surrounds lumen 166. FIG. 3C is a second embodiment of flat rings 158 that have a polyhedral surface, which is a square 168.

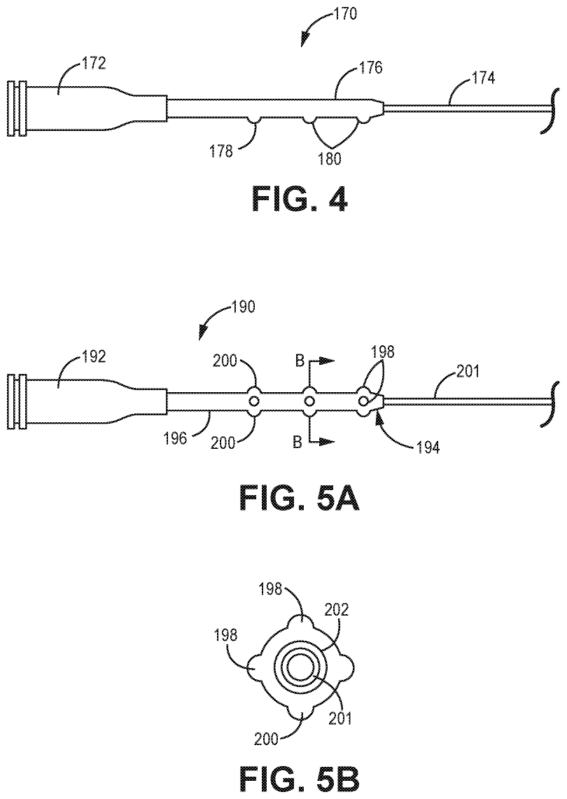

FIG. 4 depicts catheter 170 having hub 172, catheter shaft 174 and an anchoring strain relief member 176 with rounded detents 178 having tips 180. FIG. 5A depicts catheter 190 having hub 192, catheter shaft 194 and an anchoring strain relief member 196 with rounded detents 198 having tips 200. FIG. 5B depicts detents 198 spaced around a circumference of strain relief member 196 and, in this embodiment, perpendicular to each other. Catheter shaft 194 has first hollow tube 201 and second hollow tube 202 fitted over first hollow tube 201.

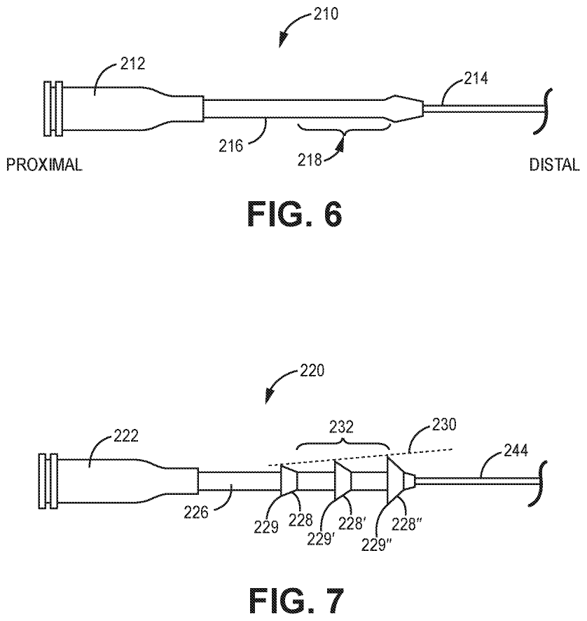

FIG. 6 depicts catheter 210 having hub 212, catheter shaft 214 and an anchoring strain relief member 216 with reverse taper 218. The taper increases in diameter from a proximal-to-distal direction. The reverse taper naturally functions as a backstop to proximal movement of the cathter relative to a valve with an elastic sealing member engaging the strain relief member. FIG. 7 depicts catheter 220 having hub 222, catheter shaft 224 and an anchoring strain relief member 226 with barbs 228, 228′, 228″ having respective tips 229, 229′, 229″ that increase in height from a proximal-to-distal direction as indicated by tangent line 230 to provide a reverse taper 232 defined by the tips of the barbs. Barbs 228, 228′ and 228″ have proximal surfaces that can function as a backstop for proximal movement of the catheter by engaging a sealing member of a valve.

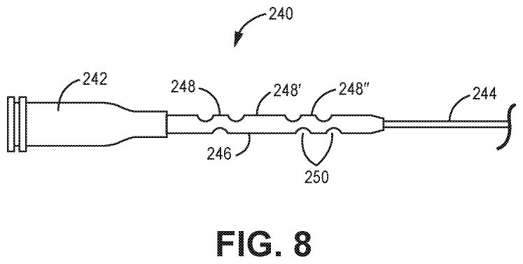

FIG. 8 depicts catheter 240 having hub 242, catheter shaft 244, and an anchoring strain relief member 246 with ridges 248, 248′, 248″ defined by notches 250. Notches 250 can also engage an elastic sealing member of a hemostatic valve to provide a backstop function.

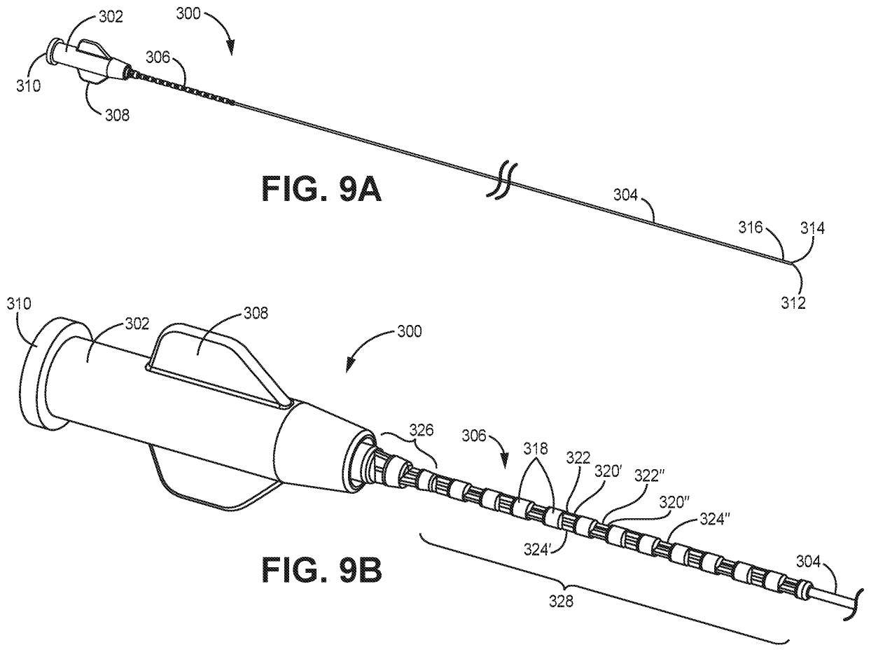

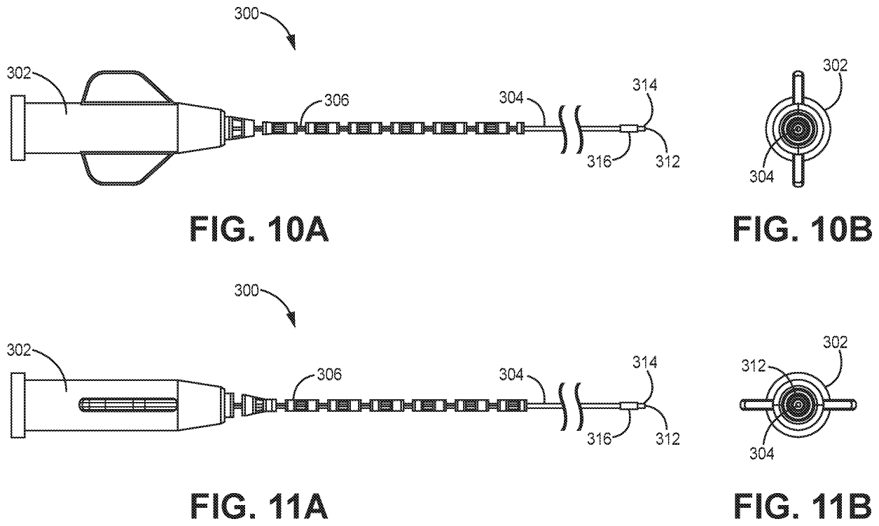

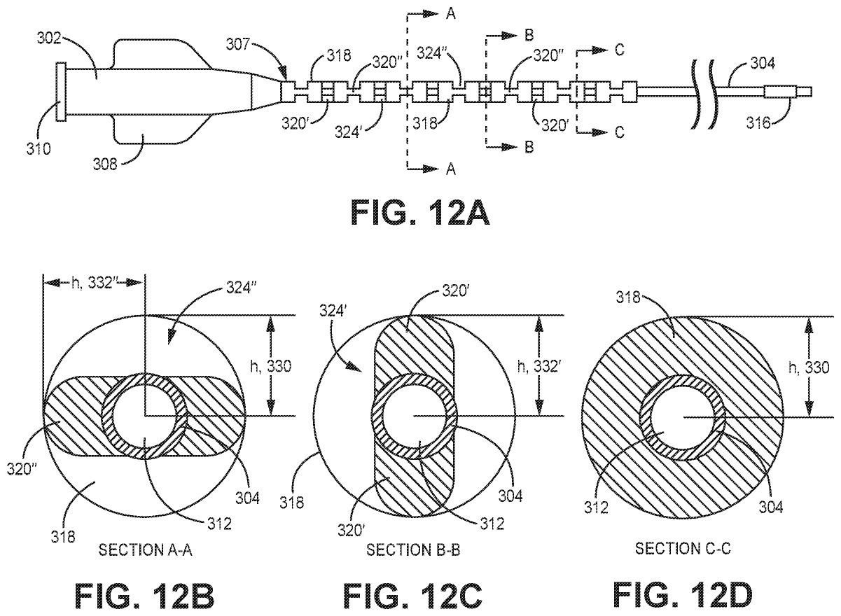

FIGS. 9A-11B depict another embodiment. Catheter 300 has hub 302, shaft 304, and anchoring strain relief member 306. Hub 302 has wings 308 and connector 310. Catheter shaft 304 has opening 312, distal tip 314, and radiopaque band 316. FIGS. 12A-12D depict an alternative embodiment of anchoring strain relief member 307. Strain relief members 306, 307 differ in the member 306 has a tapered portion 326 (FIG. 9A) that is not present in member 307. The portion 328 in FIG. 9A has a constant outer diameter, and it can be seem that portion 328 fits within an imaginary cylinder of a constant diameter that is coaxial with catheter shaft 304. Referring collectively to FIGS. 9A-12D, the anchoring strain relief members 306, 307 have cylinders 318 and ridges 320′, 320″ with respective planar surfaces 322′, 322″. Notches 324′, 324″ define ridges 320′, 320″ respectively. Heights of cylinders 318 and ridges 320′, 320″ are depicted as heights 330, 332′, 332″, respectively. The embodiment in FIGS. 9A-11B provide a continuous surface over a significant length with good texturing for gripping an elastomeric sealing ember of a hemostatic valve as well as providing a backstop function.

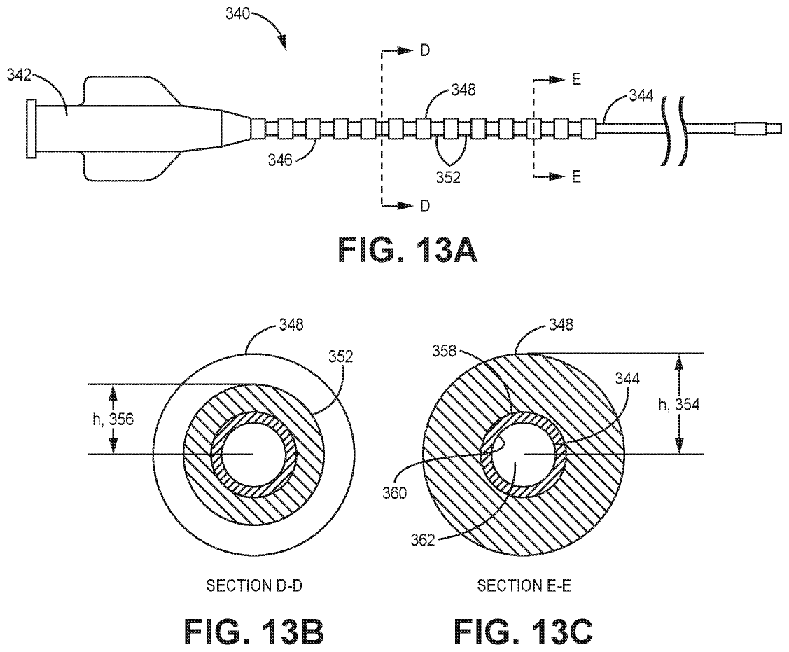

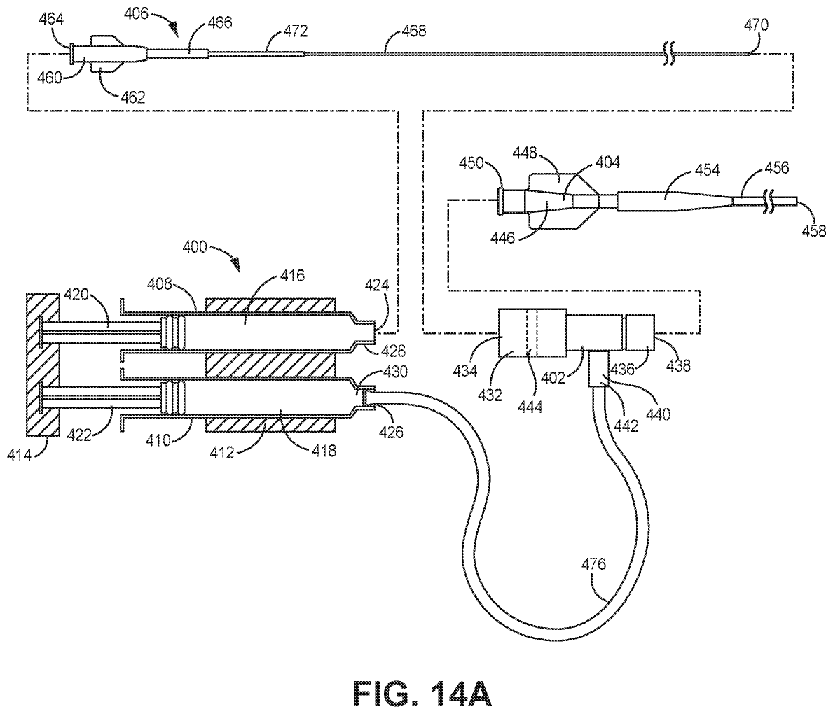

FIGS. 13A-13C depict catheter 340 having hub 342, catheter shaft 344, and an anchoring strain relief member 346 with ridges 348 defined by notches 352. Ridges 348 are cylindrical with height 354. Notches 352 provide a backstop for proximal movement of the catheter within a hemostatic valve by providing an engagement surface for an elastomeric sealing member that is below the neighboring ridges. Ridges 348 are cylindrical with height 356. Shaft 344 has outer surface 358, inner surface 360, and lumen 362. In some embodiments, ridges 348, relative to each other, can have a constant circumference and their respective heights, such that circumferences, and surface areas can be essentially equal. In this context, the term essentially equal means being within 10% of the arithmetic average of the members of the set that are being compared to each other. In one embodiment, anchoring strain relief member 346 has a length of approximately 3 cm, and a diameter of 0.13 cm (4 French). More generally, an approximately constant diameter strain relief member can have a length from about 0.5 cm to about 15 cm and in further embodiments from about 1 cm to about 12 cm, and a diameter from about 0.066 cm to about 0.34 cm and in further embodiments from about 0.1 cm to about 0.3 cm. A person of ordinary skill in the art will recognize that additional ranges of lengths and diameters within the explicit ranges above are contemplated and are within the present disclosure.

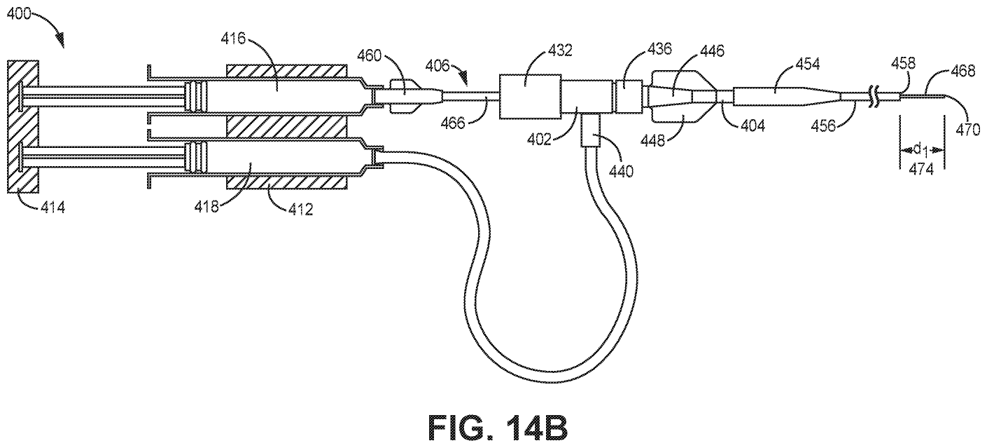

FIGS. 14A-14B depict a delivery system having dual syringe 400, connector 402, outer catheter 404, and inner catheter 406. Dual syringe 400 has first syringe 408, second syringe 410, holder 412, and grip 414. Holder 412 and grip 414 are shown conceptually in a cut-away view; artisans are familiar with providing these features. Syringes 408, 410 have respective barrels 416, 418 and plungers 420, 422, openings 424, 426, and connectors 428, 430. Connector 402 has proximal connector 432 with proximal opening 434, distal connector 436 with distal opening 438, side port 440 with side port opening 442, and sealing member 444. Sealing member 444 is sealingly disposed inside connector 402 to seal proximal opening 434 and provide for opening 442 of side port 440 and opening 438 of distal connector 436 to fluidly communicate interiorly to connector 402. Outer catheter 404 has hub 446 with wings 448, connector 450, strain relief member 454, and outer catheter shaft 456 having distal tip 458. Inner catheter 406 has hub 460 with wings 462, connector 464, anchoring strain relief member 466, and inner catheter shaft 468 having distal tip 470 and proximal hollow tube 472. Hollow tube 472 provides a thickened portion of inner catheter shaft 468. Detail for anchoring strain relief member 466 is not shown; ridges may be provided with or without notches as described elsewhere herein. When assembled, inner catheter shaft 468 may be positioned to extend beyond outer catheter shaft 456 by a distance 474. Conduit 476 fluidly joins connector 402 and dual syringe 400. The delivery system may be assembled by joining connector 402 to outer catheter 404, joining inner catheter 406 to connector 402 by passing inner catheter shaft 468 through connector 402 and sealing member 444. Dual syringe 400 is joined to connector 402 via conduit 476 and to inner catheter 406 via hub 460.

Artisans are familiar with methods for using catheters, introducing catheters into a patient and guiding catheters to deploy them at a desired location, including the placement of nested catheter systems, such as coaxial catheter systems. In an improvement adapted from such familiar methods, however, the anchoring strain relief member in the devices described herein may be used as a sealing and gripping surface. In particular, a sealing member, e.g., an elastomeric material, may be pressed against a sealing portion of the anchoring strain relief member, with the compressive member deforming to provide a seal with the sealing portion of the member, which has ridges that project into the elastomeric material to provide a resistance to movement of the member relative to the compressive material. An anchoring strain relief member has been found to be particularly useful for providing sealing and gripping when mounted on an inner catheter of a coaxial catheter system. The anchoring strain relief member may be positioned within a sealing member of a connector to provide a seal around the inner catheter. The sealing member may be an elastomeric sealing member.

Connector 402 is an example of a hemostatic valve, such as a Tuohy-Borst Adapter. These are known to artisans and are commercially available. The hemostatic valves can be opened and closed using various motions, such as sliding/snapping, movement of a lever, or rotation of a knob, and for current application, a rotating embodiment can be desirable, although any version can be used. Examples of suitable valves include, for instance, a valve with a rotatable cap, U.S. Pat. No. 4,723,550 to Bales et al., entitled “Leakproof Hemostatic Valve with Single Valve Member,” a valve with a rotating knob, U.S. Pat. No. 5,591,137 to Stevens, entitled “Hemostasis Valve with Locking Seal,” U.S. Pat. No. 5,911,710 to Barry et al., entitled “Medical Insertion Device with Hemostatic Valve,” and a valve with a first sealing member that opens upon rotation of a knob and a second sealing member that closes upon further rotation of the knob, published U.S. patent application 2018/0256872 to Agrawal et al., entitled “Hemostasis Valve and Methods for Making and Using Hemostasis Valves,” all of which are incorporated herein by reference. Such adaptors have elastomeric members that seal an opening of the adaptor. In some embodiments, the elastomeric sealing members are a membrane that is engaged to form a seal and disengaged to allow relative movement embodiments of the membrane are, for example, a membrane that is continuous or has a slit, slot or opening with various configurations available in commercial devices, and see examples below. Other embodiments of a sealing member are one or more sealing elements that engage a surface of a catheter, for instance a sealing ring. Tuohy-Borst Adapters may include a tightening feature operable to increase compression between a catheter assembly and the sealing member after the catheter assembly is in place proximate the sealing member. When interfaced with a shaft, the elastomeric member provides a seal around the shaft. Materials for the elastomeric members are known, including silicone, fluoropolymers, rubbers or the like. The connector, such as a Tuohy-Borst Adapter, may optionally comprise an actuating member that is movable, e.g. by rotation, to provide a further compressive force to the elastomeric member (e.g., FLO 40 Tuohy-Borst Adapter, Merit Medical, Salt Lake City, Utah). Tuohy-Borst Adapters are available with or without a side port. If a Tuohy-Borst Adapter is used without a side port, a further connector that has a side port may be used in a nested, e.g. coaxial, catheter system by, for example, placing the further connector between the Tuohy-Borst Adapter and the outer catheter. Fluid conduits to the delivery system may then be joined as appropriate to establish communication with the inner catheter and/or outer catheter. References to connecting connectors in a catheter system refer to establishing a fluid-tight communication and may be a direct connection or an indirect connection unless otherwise specified.

In general, the insertion of the catheter with the sealing strain relief member through a hemostatic valve and sealingly securing the strain relief member in the valve can provide particularly useful configurations for the delivery of an inner catheter within an outer catheter. Such a configuration is generally referred to herein as a nested catheter configuration for convenience. If the outer catheter is cylindrically symmetric, single lumen catheter, the nested configuration can be referred to as being coaxial even if not constrained to be precisely coaxial, but the nested catheters do not need to be coaxial. In general, the use of nested catheters can be convenient and useful for a variety of medical procedures, and the lengths and diameters of the catheters can be selected to be suitable for the specific procedures. The catheters herein with sealing strain relief members can generally be used in these various procedures. Referring to FIGS. 14A and 14B, a more detailed embodiment is described above relating to the delivery of separate fluids through the nested catheter for combining the fluids at the distal ends of the catheter, but this detailed discussion is not intended to suggest anything more than this embodiment being of some particular interest.

Dual syringe system 400 is a dual syringe system and is an embodiment of a delivery system. A delivery system may provide for removal, withdrawal, or both, of materials via the catheter lumen. For instance, a peristaltic pump may be used instead of a syringe or a syringe pump may be used instead of a manually operated dual syringe system. Other flow systems are known and may be used with the catheters. Similarly, delivery systems that withdraw fluids and/or other materials using a syringe, a pump, or other means are known and may be used.

Catheters comprise a hollow tube that provides the catheter shaft. A hub is attached to the catheter at the proximal end of the catheter. A distal end of the catheter is the end that is introduced into a patient. The invention is suited for use with various catheter lengths and diameters, for example, medical catheters of at least 10 cm in length and no more than 12-160 cm; artisans will immediately appreciate that all ranges and values between the explicitly stated bounds are contemplated, with 10, 12, 15, 20, 25, 35, 40, 50, 75, 100, 125, 150, 160 cm being available as a lower or an upper limit. Catheter inner and outer diameters, for example, may be from 0.2-10 mm; artisans will immediately appreciate that all ranges and values between the explicitly stated bounds are contemplated, e.g., 0.2, 0.4, 0.6, 0.8, 1, 1.1, 1.2, 1.3, 1.4, 1.5, 1.6, 1.7, 1.9, 2, 3, 4, 4.5, 5, 10 mm being available. An inner diameter is necessarily less than the outer diameter. Further, artisans are able to choose inner and outer diameters for a plurality of nested catheters that are to be used with an inner catheter having an outer diameter that is capable of passing through an inner diameter of the outer catheter. The catheter may have a constant shaft inner and outer diameter and connect directly to a hub or the shaft inner and/or outer diameter may be varied at all or a portion of the shaft. A catheter shaft that has an increased wall thickness at its proximal end may be useful in conjunction with a strain relief member and may underlie all or a portion of the strain relief member and/or extend beyond the strain relief member. For instance, a second hollow tube may be overlaid over a smaller hollow tube to provide the catheter shaft.

Many materials for catheters are known, including, e.g, one or more biocompatible materials, including, for example, metals, such as stainless steel or alloys, e.g., Nitinol®, or polymers such as polyether-amide block co-polymer (PEBAX®), nylon (polyamides), polyolefins, polytetrafluoroethylene, polyesters, polyurethanes, polycarbonates, polysiloxanes (silicones), polycarbonate urethanes (e.g., ChronoFlex AR®), mixtures thereof, or other suitable biocompatible polymers. Radio-opacity can be achieved with the addition of metal markers or plastics loaded with dense materials (i.e. metallic or mineral powders), which can be made from gold, platinum-iridium, radiopaque compounds or other suitable elements. Catheter bodies can be extruded or formed through other appropriate polymer processes. Catheter walls can include fine metal reinforcements that can be melted into the polymer or otherwise processed for embedding into the polymer, such as with polymer shrink wrap. Fittings and the strain relief member can be overmolded onto the catheter shaft or otherwise heat bonded, adhesive bonded, or the like, or combinations thereof.

The inventors determined that an anchoring strain relief member could be made that fills a role of strain relief for a catheter but further provides an anchoring feature. The anchoring feature provides for higher pressures to be used in the catheter because it provides a better seal than a catheter shaft, thus resisting the linear forces created at higher pressures that may unseat the catheter. A higher pressure is useful not only for a rate of fluid movement but also for moving high viscosity materials, or for using a smaller diameter catheter than would otherwise be suitable.

The anchoring strain relief member may be made of a plurality of pieces or may be monolithic, meaning made of a single continuous piece. The strain relief member may be molded in place, formed with a catheter, or separately formed followed by attachment to the catheter shaft, such as with thermal bonding, adhesive bonding, or other suitable approach. Materials for use in the strain relief member may be, for instance, metal, elastomers, thermoplastics, thermoset plastics, silicones, fluoropolymers, combinations thereof, and the like.

The anchoring strain relief member may have a sealing portion that is intended for sealing with an elastomeric member and another portion that is not. For instance, the embodiment of FIG. 9B has a tapered portion 326 with an outer diameter that is not suited to placement within a Tuohy-Borst sealing member. A portion that is suited for sealing preferably is not tapered, meaning it is not tapered in a proximal-to-distal direction. Alternatively, a taper in a proximal-to-distal direction has a taper angle of no more than about 5 degrees, in further embodiments no more than about 3 degrees, and in additional embodiments no more than about 1.5 degrees. A person of ordinary skill in the art will recognize that additional ranges of taper angles within these explicit ranges are contemplated and are within the present disclosure. A reverse taper can be useful since a reverse taper can provide some backstop function itself and naturally forms at least one ridge through the reverse taper. It is also useful to seal around a portion of the member that has ridge heights that are essentially equal to each other.

An anchoring strain relief member may have a surface that comprises a plurality of ridges. A ridge is an elevated body part or structure. Dimensions of ridges are measured in terms of a perpendicular distance to a center of the catheter's lumen unless otherwise specified, e.g., see FIGS. 12A-13C. The ridges resist movement of the anchoring strain relief member when it is engaged with a compressive force from an elastomeric member or other source. Surface texturing can complement the sealing efficacy provided by the ridges, as described further below. Ridges may be distributed so that a plurality of the ridges or a predetermined number of ridges are covered by an elastomeric member used to seal around the ridges. Accordingly, embodiments include a predetermined number of ridges per mm of length, referred to herein as a linear density, with the length being taken on the outer surface of the member for a distance that is parallel to the lumen central axis with the ridge number per millimeter (mm) being from 0.2-20; artisans will immediately appreciate that all ranges and values between the explicitly stated bounds are contemplated, e.g., 0.2, 0.5, 1, 2, 3, 4, 5, 6, 7, 8, 9, 10, 15, 20 ridges per mm. The number of ridges per mm may advantageously be used to control a resistance to movement of a catheter, particularly an inner catheter of a coaxial catheter system wherein movement of the inner catheter relative to one or more other catheters is desired. Ridge heights are chosen in light of considerations such as a desired pull-out strength, a linear density of the ridges, fill-volume, and dimensions of the catheter that has the anchoring strain relief member. Ridge heights may be, for example from 0.2-5 mm; artisans will immediately appreciate that all ranges and values between the explicitly stated bounds are contemplated, e.g., 0.2, 0.3, 0.4, 0.5, 1, 1.5, 2, 2.5, 3, 4 mm. Similarly, a difference between a ridge tip height and a radius of an outer catheter surface to which to strain relief member is attached may be from 0.1-4 mm; artisans will immediately appreciate that all ranges and values between the explicitly stated bounds are contemplated e.g., 0.1, 0.2, 0.3, 0.4, 0.5, 1, 1.5, 2, 2.5, 3, 4 mm. A radius of a catheter and/or catheter surface is measured from the catheter's central axis.

Spaces between ridges are referred to as notches and certain embodiments include an anchoring strain relief member that has a surface that comprises a plurality of notches. In one embodiment, the member has a constant circumference and height except for the notches, with the notches having a depth. The notches may be independently selected to have a depth from 0.05-4 mm; artisans will immediately appreciate that all ranges and values between the explicitly stated bounds are contemplated, e.g., 0.05, 0.1, 0.2, 0.3, 0.4, 0.5, 1, 1.5, 2, 2.5, 3, 4 mm.

Another metric to quantify characteristics of ridges and/or notches is volumetric. A portion of an anchoring strain relief member is placed in an imaginary cylinder of constant diameter that is concentric with a catheter lumen, with the imaginary cylinder being tangent to a ridge at a proximal end of the cylinder and at a distal end of the cylinder; this volumetric metric is not used when these criteria are not applicable and may be applied to an entire member or to only a portion of an anchoring strain relief member; further a length of the imaginary cylinder is at least 0.1 mm and the length may be specified as a value or range from 0.1 mm-10 cm, e.g., 0.01 mm, 0.25 mm, 0.5 mm, 1 mm, 2, mm, 5 mm, 7.5 mm, 1 cm, 2.5 cm, 5 cm, 7.5 cm, or 10 cm. The member is solid and occupies a percentage of the cylinder's volume. This metric is referred to as a fill-volume. Embodiments include an anchoring strain relief member having a fill-volume from 50-90%; artisans will immediately appreciate that all ranges and values between the explicitly stated bounds are contemplated, e.g., 50, 60, 70. 80. 90%.

Since the anchoring strain relief member provides a seal with an elastomeric member, it is part of, or attached to, the catheter so that there is a fluid-tight seal between the strain relief member and elastomeric sealing member. Further, the member is completely free of, or has at least a sealing portion that is free of, any channel that would provide for a flow of fluid from a distal end to a proximal end of the member when the member is in a sealing position with an elastomeric member. Such channels are referred to as fluid channels herein. Being free of fluid channels allows for the creation of a seal. A portion of an anchoring strain relief member that is free of fluid channels may be, for example, from 1-15 cm, artisans will immediately appreciate that all ranges and values between the explicitly stated bounds are contemplated, e.g., 1, 2, 3, 4, 5, 6, 8, 10, 12, 14, 15 cm.

Examples of ridges and/or notches are provided in FIGS. 1A-13C. Ridge and/or notch heights, linear densities, and fill-volumes are described above and are applicable generally and to the embodiments specifically described herein. FIGS. 1A-1B depict barbs. Barbs have a tapered projection that tapers from a large height proximally to a lesser height distally. FIG. 2 depicts circular rings that each have a constant height that is essentially equal to the other depicted rings. The rings have rounded surfaces. FIGS. 3A-3C depict flat rings that are rounded or squared. The rings each extend around an entire circumference of the member and have a height at every point in the circumference. Alternative embodiments provide for the ring shapes and/or heights to be selected independently or to be tapered. FIGS. 4-5B depict various detents. Detents are projections that do not extend around an entire circumference of the member. FIGS. 6 and 7 depict different embodiments of an anchoring strain relief member that has a reverse taper. The barbs of FIG. 7 extend around a circumference of the member. FIG. 8 depicts an anchoring strain relief member with sealing and gripping features that are readily described in terms of notches relative to an outermost radius of the strain relief member, which is constant in the sealing portion in the depicted embodiment. The notches may be selected as described elsewhere herein and the sealing portion of the strain relief member may be constant, varies, or have a reverse taper. FIGS. 9A-13C depict strain relief members that comprise a length having a plurality of rings separated by notches. The term ring is broad and includes ridges that extend around a perimeter of a transverse cross-section of the member, for example, cylinders, right cylinders, cuboids, and cones. In FIGS. 9B-12D the notches comprise planar surfaces, with adjacent notches having planar surfaces at right angles to each other. Offsetting the planar surfaces at 10-90 degrees relative to each other advantageously changes vectors of forces impinging on the member in adjacent notches to increase a resistance to pull. Artisans will immediately appreciate that all ranges and values between the explicitly stated bounds of 10-90 degrees are contemplated, e.g., 10, 20, 30, 40, 45, 50, 60, 70, 80, 90 degrees.

Catheters that comprise an anchoring strain relief member are not limited as to size, however the member has been observed to be particularly useful on an inner catheter of a nested catheter system with the inner catheter having an outer diameter from 0.2-3 mm; artisans will immediately appreciate that all ranges and values between the explicitly stated bounds are contemplated, e.g., 0.2, 0.3, 0.4, 0.5, 0.6, 0.8, 1, 1.2, 1.; 1.4, 1.5, 1.6 2, 2.2, 2.4, 2.6, 2,8, 3 mm. Artisans are familiar with medical catheters and will recognize the scope and bounds of this term. Medical catheters are sterilizable and/or may be provided in a sterilized form, e.g., in packaging that accommodated their use with sterile technique.

Kits and systems are useful for providing catheters comprising an anchoring strain relief member matched to a hemostatic valve, such as a Tuohy-Borst Adapter, for efficient sealing and pull-out forces. Additionally or alternatively, a system may further comprise the outer catheter and/or other components such as fluid delivery components, other fittings or further medical devices for use with or delivery through the catheter. The adaptors may be provided with standardized connections for ready connection to variously sized outer catheters. The anchoring strain relief member may be an embodiments provided herein; catheters and Tuohy-Borst Adapters may be chosen from any source provided they do not prevent operation of the anchoring strain relief member embodiment. The various components of systems may or may not be commonly packaged. Also, various components can be provided in ranges of sizes that may be differently selected for particular patients.

As noted above, an objective of the presently described catheters is directed to the ability of the anchoring strain relief member to engage a hemostatic valve with sufficient stability to withstand greater amounts of pressure without disengaging. In the Examples below, testing is performed to quantify this sealing ability. Using pull out force measurements using a universal tester with a gantry speed of 300 mm/min, the pull out force can be measured and converted to a pressure value. With the catheter embodiments described herein, the pull out force expressed as a pressure can be at least 9 N, in further embodiments at least about 10 N and in additional embodiments at least about 12 N. A person of ordinary skill in the art will recognize that additional ranges of pressures within the explicit ranges above are contemplated and are within the present disclosure.

All patents, publications, and references provided in this patent application are hereby incorporated by reference herein for all purposes; in case of conflict, the instant specification is controlling.

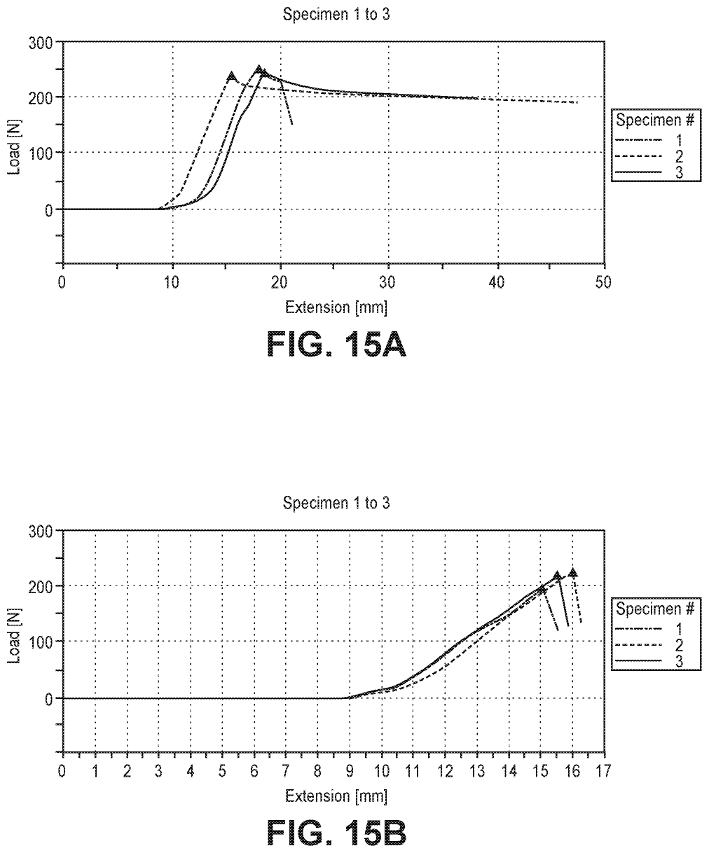

Example 1 describes an embodiment of a catheter equipped with an inventive anchor strain relief member. Example 2 describes back pressure force testing. The anchoring strain relief member required an average 246 N (1,779 PSI) to dislodge the strain relief member under the backpressure test conditions, FIG. 15A, compared to 214 N (1,547 PSI) for a conventional strain relief member, FIG. 15B. The anchoring strain relief member slowly moved and stopped moving without exhibiting elongation once backpressure was released. In contrast, the conventional strain relief member exhibited stretching and ultimately was rapidly ejected from the assembly. The backpressure test measured backpressure by observing the force applied to a plunger of a 1 ml syringe that provided water for the backpressure. As is evident, the anchoring strain relief member provided a much greater resistance. Example 2 describes pull-out force testing when the anchoring strain relief member is in a sealing position in a Tuohy-Borst Adapter. The pull-out force was 15 N compared to 7 N with a conventional strain relief member. These Examples demonstrate the very superior anchoring properties of the anchoring strain relief member. These are useful for applying increased pressure to materials passed through a catheter assembly and also to the user that manipulates the catheter assembly. Further, the increased resistance, resistance to stretching, and maintenance of sealing integrity is useful for fine-tuning positioning of the catheter when in use. Moreover, it is believed that the inventors are the first to make and use a strain relief member as a sealing member.

### EXAMPLES

### Example 1

### Strain Relief Anchor Member

The catheter with the strain relief anchor member of FIGS. 9A-11B was prepared. The catheter was a stainless-steel coil-reinforced polyamide shaft having inner and outer diameters of 0.014 inch and 0.017 inch, respectively, and having the strain relief and hub assembly adhered to the proximal end. The hub assembly was a luer hub meeting ISO 80369-7 (2016) standards.

Strain relief member 306 was prepared by overmolding a thermoplastic elastomer onto the catheter shaft. Strain relief member cylinders 318 had a diameter of 0.051 inches and ridges 320 had a maximum diameter of 0.051 inches and a thickness of 0.015 inches relative to the catheter outer surface. Portion 328 had a length of 3 cm.

### Example 2

### Back Force Pressure Testing

This test measured the force required to dislodge the strain relief anchor from a Tuohy-Borst Adapter. A commercial Tuohy-Borst Adapter having elastomeric circumferentially sealing member with a proximate opening of 0.053 inches, a side-port opening and a distal opening prepared with a dead-end cap to prevent fluid from exiting the distal opening. A 1 ml syringe containing water was connected to the Tuohy-Borst Adapter side port. The catheter of Example 1 was cut down in length and passed through the sealing member of the hemostatic adapter and positioned with the anchoring strain relief member in contact with the sealing member. The distal end of the catheter was blocked so as not to pass fluid. The assembly was arranged in an Instron® (3343 model no) universal tester to measure the force required to depress the plunger of the 1 ml syringe. A comparison assembly with the same dimensions was prepared except using a standard (smooth) strain relief.

A gantry travel speed of 300 mm/min was applied and the force on the plunger was measured, FIG. 15A (anchor strain relief assembly) and FIG. 15B (comparison assembly). Three trials were made for each assembly. The anchor strain relief assembly dislodgement force mean value was 1,779 PSI, standard deviation 45 PSI; the maximum force was 1,822 PSI with a range of 87 PSI. The comparison assembly dislodgement force mean value was 1,547 PSI, standard deviation 112 PSI; the maximum force was 1,634 PSI with a range of 210 PSI.

### Example 3

### Pull-Out Force Testing

This test measured the force required to pull an anchoring strain relief member from a hemostatic adapter. The anchoring strain relief member and comparison relief member assemblies were prepared as in Example 2. Each were placed in a commercial Tuohy-Borst Adapter and mounted in an Instron® (model 3343) tester with the Tuohy-Borst Adapter held in a fixed position and the gantry fixed to the proximal end of the catheter. Gantry travel speed was set to 300 mm/min.

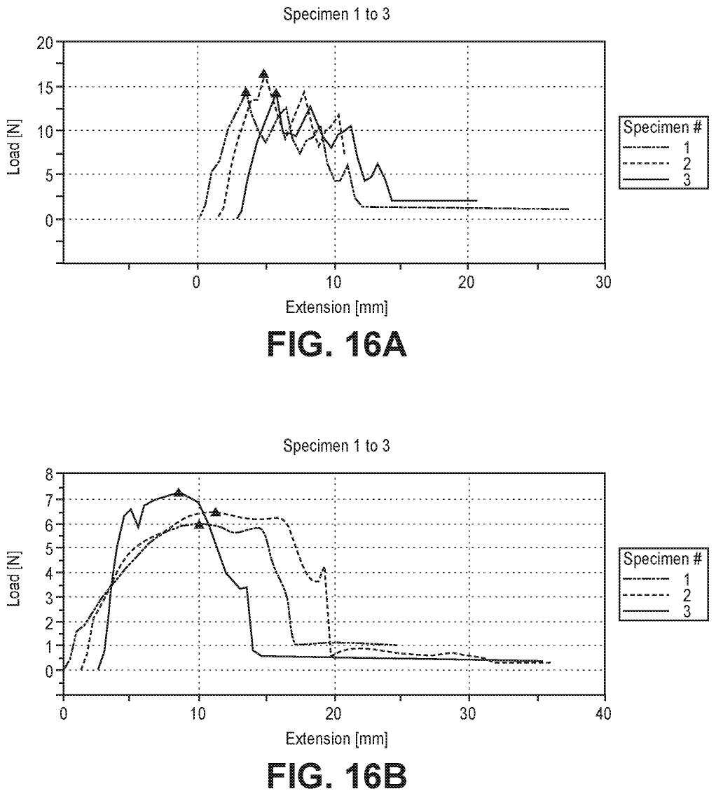

The anchoring strain relief member pull out force was an average (3 trials) 15 N with a standard deviation of 1.2, maximum of 16.6 and range of 2.2, FIG. 16A. The conventional comparison strain relief member pull out force was an average (3 trials) 7 N with a standard deviation of 0.6, maximum of 7.3 and range of 1.3, FIG. 16A.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understand that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated. The use of the term “about” herein refers to measurement error for the particular parameter unless explicitly indicated otherwise.

What is claimed is:

1. A medical catheter having a proximal end and a distal end, the catheter comprising
a catheter shaft having a catheter lumen, a catheter inner surface, and a catheter outer surface separated from the catheter inner surface by a catheter wall thickness,
a hub attached to the proximal end of the catheter shaft, and
an anchoring strain relief member distal to the hub, joined to the catheter outer surface, and comprising a sealing portion that comprises at least one ridge that has a ridge tip and a ridge height defined by a distance from the ridge tip to the catheter central axis, the distance being measured perpendicular to the central axis, wherein the ridge forms a flow barrier between the catheter outer surface and the top of the ridge and wherein if the sealing portion comprises a plurality of ridges each having a ridge tip and a ridge height, then a set of the ridge tips have no taper, a reverse taper, or no more than a 5 degree forward taper in a proximal to distal direction.

2. The medical catheter of claim 1 wherein the sealing portion has no taper.

3. The medical catheter of claim 1 wherein the sealing portion is free of fluid channels.

4. The medical catheter of claim 1 wherein each of the plurality of ridges have ridge tip heights that are essentially equal.

5. The medical catheter of claim 1 wherein the sealing portion has a longitudinal length from 1-10 cm.

6. The medical catheter of claim 1 wherein at least three of the plurality of ridges define a first, a second, and a third ring, with a first planar surface separating the first and the second rings and a second planar surface separating the second and the third rings, wherein the first planar surface and the second planar surface are parallel to the catheter central axis and offset relative to each other.

7. The medical catheter of claim 6 wherein the first planar surface and the second planar surface are perpendicular to each other.

8. The medical catheter of claim 6 wherein at least two of the rings define cylinders of constant circumference.

9. The medical catheter of claim 1 wherein at least two of the ridges each define a barb with a base of the barb being distal relative to a point of the barb.

10. The medical catheter of claim 1 wherein at least two of the ridges each define a ring.

11. The medical catheter of claim 1 wherein the strain relief member comprises one or more notches that are positioned such that engagement of a notch by an elastic sealing member of a valve positions a ridge to act as a backstop to proximal movement of the catheter relative to the valve.

12. The medical catheter of claim 1 wherein at least two of the ridges each define a detent.

13. The medical catheter of claim 1 wherein the ridge heights define a taper or a reverse taper with a taper slope of no more than 5%.

14. The medical catheter of claim 1 wherein the sealing portion has an essentially constant outer diameter and the plurality of ridges are defined by a plurality of notches in the anchoring strain relief member.

15. The medical catheter of claim 1 wherein the anchoring strain relief member comprises an elastomeric material.

16. The medical catheter of claim 1 wherein the ridge heights range from about 0.2 mm to about 3 mm.

17. The medical catheter of claim 1 wherein differences between the ridge heights of the plurality of ridges and a radius of the catheter outer surface are in a range from about 0.05 mm to about 3 mm.

18. The medical catheter of claim 1 wherein the plurality of ridge heights is a number from 2-50.

19. The medical catheter of claim 1 wherein a linear density of the plurality of ridge heights is from 0.2-5 per mm.

20. The medical catheter of claim 1 having a diameter of the catheter shaft outer surface from about 0.2 mm to about 3 mm.

21. A method for using the medical catheter of claim 1, the method comprising delivering a substance through the catheter.

22. The method of claim 21 wherein the sealing portion provides a seal against an elastomeric sealing member of a hemostatic valve.

23. A method of assembling a nested catheter system comprising
providing an outer catheter that comprises:
an outer catheter hub and an outer catheter shaft comprising an outer catheter lumen, an outer catheter inner surface, and an outer catheter outer surface, with the outer catheter hub being connected to the an outer catheter shaft to provide fluid communication between the outer catheter hub and the outer catheter lumen;
providing an inner catheter that comprises:
an inner catheter hub, an anchoring strain relief member, and an inner catheter shaft comprising an inner catheter lumen with a central axis, an inner catheter inner surface, and an inner catheter outer surface, with the inner catheter hub being connected to the inner catheter shaft to provide fluid communication between the inner catheter hub and the inner catheter lumen, with the anchoring strain relief member being sealingly joined to the inner catheter outer surface;
providing a connector that comprises a first opening and an elastomeric sealing member, with the sealing member providing a seal across the first opening;
attaching the connector to the outer catheter hub in fluid communication with the outer catheter lumen and with a second opening between the connector and the outer catheter lumen,
passing the inner catheter shaft through the first opening and the sealing member and into the outer catheter shaft lumen, with the connector being in fluid communication through the second opening with a annulus formed between the inner catheter outer surface and the outer catheter inner surface, and
positioning a sealing portion of the strain relief member within the sealing member, with the sealing member pressing against the portion of the strain relief member to establish a seal.

24. The method of claim 23 wherein the connector is a hemostatic valve.

25. The method of claim 23 wherein the hemostatic valve is a Tuohy-Borst Adapter with a side opening.

26. The method of claim 23 further comprising delivering a fluid through the lumen.

27. The method of claim 23 wherein the strain relief member comprises a sealing portion that comprises at least one ridge that has a ridge tip and a ridge height defined by a distance from the ridge tip to the catheter central axis, the distance being measured perpendicular to the central axis, wherein the ridge forms a flow barrier between the catheter outer surface and the top of the ridge and wherein if the sealing portion comprises a plurality of ridges each having a ridge tip and a ridge height, then a set of the ridge tips have no taper, a reverse taper, or no more than a 5 degree forward taper in a proximal to distal direction.

28. A system comprising a hemostatic valve and a medical catheter comprising an anchoring strain relief member comprising an elastomeric polymer and having a sealing portion, wherein the hemostatic valve comprises a connector and a sealing member, wherein the sealing portion of the anchoring strain relief member can be engaged by the sealing member of the hemostatic valve to form a fluid tight seal.

29. The system of claim 28 wherein the medical catheter has a proximal end and a distal end, the catheter comprising
a catheter shaft having a catheter lumen, a catheter central axis, a catheter inner surface, and a catheter outer surface separated from the catheter inner surface by a catheter wall thickness,
a hub attached to the proximal end of the catheter shaft, and
the anchoring strain relief member is distal to the hub, sealingly joined to the catheter outer surface, and comprises the sealing portion that comprises a plurality of ridges that each have a ridge tip that has a ridge height as defined by a distance from the ridge tip to the catheter central axis, the distance being perpendicular to the central axis.

30. The system of claim 28 further comprising a second catheter comprising an outer catheter hub with a connector and an outer catheter shaft having an outer catheter lumen, wherein the outer catheter hub is configured to engage the connector of the hemostatic valve and wherein the outer catheter lumen has a dimensions allowing for the passage of the medical catheter shaft.

31. The system of claim 28 wherein the sealing portion that comprises a plurality of ridges engages the sealing member of the hemostatic valve in a fluid tight seal at a sealing zone with a length along the catheter surface of at least about 0.1 mm wherein each ridge forms a flow barrier between the catheter outer surface and the top of the ridge and wherein and has a ridge tip and a ridge height, such that a set of the ridge tips have no taper, a reverse taper, or no more than a 5 degree forward taper in a proximal to distal direction.
